(19) United States
(12) Patent Application Publication
(10) Pub. No.: US 2024/0298665 A1
(43) Pub. Date: Sep. 12, 2024

(54) CHEWING GUM COMPOSITIONS FOR SKIN BENEFITS

(71) Applicant: WM. WRIGLEY JR. COMPANY, Chicago, IL (US)

(72) Inventors: Grace LYU, Guangdong (CN); Ken YI, Guangdong (CN); Melanie CHU, Chicago, IL (US); Holly KNUTSEN, Chicago, IL (US)

(21) Appl. No.: 18/549,469

(22) PCT Filed: Mar. 7, 2023
(86) PCT No.: PCT/CN2021/079458

§ 371 Date: Sep. 7, 2023

(51) Int. Cl. A23G 4/14; A23G 4/06; A23G 4/08; A23G 4/12; A23G 4/20; A61K 8/02; A61Q 19/08

(52) U.S. Cl. CPC A23G 4/14; A23G 4/062; A23G 4/08; A23G 4/126; A23G 4/20; A61K 8/0233; A61Q 19/08; A23G 2200/04; A61K 2800/592

(57) ABSTRACT

Chewable compositions or specifically chewing gum contains a combination of beauty ingredients that are useful for protecting and/or maintaining healthy skin. More particularly, the chewing gum comprises a combination of collagen, Vitamin C, and Vitamin E, which may be useful for maintaining skin elasticity and protecting the skin from oxidative stress.

### BACKGROUND OF THE INVENTION

The current application is directed to chewable compositions, and specifically to chewing gum that contains a combination of beauty ingredients that are useful for protecting and/or maintaining healthy skin. More particularly, the chewing gum comprises a combination of collagen, Vitamin C, and Vitamin E, which may be useful for maintaining skin elasticity and protecting the skin from oxidative stress.

Collagen peptides are widely used in health food, beauty cosmetics and medical health products, and are known to provide physiological benefits, such as anti-oxidation, anti-radiation, anti-aging, calcium supplementation and calcium protection to promote bone development, improve human immunity, and promote skin cell regeneration. However, many beauty regimens are both expensive and time consuming to perform. It would thus be desirable to provide a consumer friendly delivery vehicle for beauty ingredients, such as collagen, which is easy and pleasurable to use, and may be used multiple times a day.

Using chewing gum as a carrier to deliver collagen can facilitate the intake of collagen peptides and other beauty or health ingredients for all types of people and promote health. However, current chewing gum products contain either no collagen, or collagen in low amounts. Accordingly, it would be desirable to provide a consumer friendly delivery vehicle which can deliver higher amounts of collagen and other health and beauty ingredients to consumers.

### BRIEF DESCRIPTION OF THE INVENTION

The current application is directed to chewable compositions, and specifically to chewing gum that contains a combination of beauty ingredients that are useful for protecting and/or maintaining healthy skin. More particularly, the chewing gum comprises a combination of collagen, Vitamin C, and Vitamin E, which may be useful for maintaining skin elasticity and protecting the skin from oxidative stress.

In one aspect, the present disclosure is directed to a coated chewing gum comprising: Vitamin C; Vitamin E; and collagen in an amount of from about 6% to about 20% by weight of the coated chewing gum.

In another aspect, the coated chewing gum comprises a coating and a chewing gum center.

In another aspect, the chewing gum center comprises collagen in an amount of from about 8% to about 25% by weight of the chewing gum center and Vitamin C in an amount of at least 0.1% by weight of the chewing gum center.

In another aspect, the coating comprises Vitamin E.

In another aspect, the coated chewing gum is a sugarless chewing gum.

### DETAILED DESCRIPTION OF THE INVENTION

The current application is directed to chewable compositions, and specifically to chewing gum that contains a combination of beauty ingredients that are useful for protecting and/or maintaining healthy skin. More particularly, the chewing gum comprises a combination of collagen, Vitamin C, and Vitamin E, which may be useful for maintaining skin elasticity and protecting the skin from oxidative stress.

Advantageously, the chewing gum of the present disclosure contains higher levels of collagen than is typically present in chewing gum compositions, while still maintaining good mouthfeel properties. As demonstrated in the examples, the chewing gums of the present disclosure have good shelf-life, and a high release-rate for the collagen. The chewing gums of the present disclosure are thus an effective and convenient delivery vehicle for ingredients that are useful in promoting skin health.

Thus, in one aspect, the present disclosure is directed to a chewing gum, and in particular a coated chewing gum, comprising Vitamin C; Vitamin E; and collagen in an amount of about 6% to about 20% by weight of the coated chewing gum. In certain aspects, the chewing gum is a coated chewing gum comprising a chewing gum center and a coating. The coating may completely or partially coat the exterior surface of the chewing gum center. In such embodiments, the chewing gum center comprises the collagen and Vitamin C, which is a water soluble antioxidant, and the coating comprises Vitamin E, which is a water insoluble antioxidant.

### Collagen

Collagen (also referred to herein as “collagen peptide”) is a small molecule biologically active peptide made from animal skin and bone collagen after moderate enzymolysis and refinement by edible protease. In certain instances, collagen may have significant beautifying and bone-building effects. In one aspect, at least 90% by weight of the collagen used in the chewing gum of the present disclosure has a molecular weight of less than 3,500 Da. An example of a suitable collagen for use in the chewing gums of the present disclosure is described in U.S. Pat. No. 9,072,724 and CN103209678A, which are herein incorporated by reference in their entirety.

In one embodiment, a suitable collagen for use in the chewing gums of the present disclosure is Verisol® collagen peptide, which is available from Gelita (Germany). The Verisol® collagen peptide is a small molecule and is easy to adsorb, and has an average molecular weight of 2000 Da. Studies have demonstrated that consuming 2500 mg of Verisol® collagen peptide a day can increase skin elasticity by 7% within 4 weeks, as compared with experimental control (see “Oral Supplementation of Specific Collagen Peptides Has Beneficial Effects on Human Skin Physiology: A Double-Blind Placebo-Controlled Study,” Skin Pharmacol. Physiol., Proksch, et al., 2014, 27:47-55), and eye wrinkles can be reduced by 32% within 8 weeks, as compared to experimental control (see “Oral Intake of Specific Bioactive Collagen Peptides Reduces Skin Wrinkles and Increases Dermal Matrix Synthesis,” Proksch, et al., Skin Pharmacol. Physiol., 2014, 27:113-119).

In one embodiment, the chewing gum of the present disclosure is a coated chewing gum comprising a chewing gum center and a coating that partially or completely coats the surface of the chewing gum center. The term “coated chewing gum” is used interchangeably herein with the terms “finished chewing gum” or “finished product”. The coated chewing gum (comprising a chewing gum center and a coating) may comprise collagen in an amount of from about 6% to about 20% by weight of the coated chewing gum, including from about 6.1% to about 18.5% by weight of the coated chewing gum. In one aspect, the coated chewing gum comprises, per piece of chewing gum, from about 125 to about 600 mg of collagen, including from about 150 to about 500 mg of collagen. A chewing gum of the present disclosure, e.g., a piece of chewing gum, can take a variety of shapes and forms, for example, a pellet, a gumball, a square, a stick, etc.

As discussed herein, the collagen is present in the chewing gum center. Thus, in another aspect, the chewing gum center comprises collagen in an amount of from about 8% to about 25% by weight of the chewing gum center, including from about 8.33% to about 25% by weight of the chewing gum center, or from about 8.33% to about 16.67% by weight of the chewing gum center. In another embodiment, the chewing gum center comprises, per piece, from about 125 to about 600 mg of collagen, including from about 150 to about 500 mg of collagen.

In one particular embodiment, multiple pieces of chewing gum, as described herein, may be packaged in a can for sale to consumers. In one embodiment, such a can may provide about 2840 mg of collagen per can (e.g., for a net weight of 40 g/can).

The number of pieces of chewing gum used by a consumer per day may vary, depending on the desired amount of collagen intake. In one embodiment, the consumer may use at least 1, at least 2, or at least 3 pieces of chewing gum per day. In one particular embodiment, a consumer may use 3 pieces of chewing gum a day. In one such embodiment, a consumer may use per day 3 pieces of chewing gum containing about 473 mg of collagen per piece. In another embodiment, a consumer may use per day 3 pieces of chewing gum containing about 170 mg of collagen per piece.

Advantageously, collagen is released from the chewing gum of the present disclosure at a release rate that allows for increased amounts of collagen to be delivered to a consumer upon gum chewing. As used herein, the term “collagen release rate” (or chew-out rate) refers to the percentage of collagen in the chewing gum that is released from the chewing gum after chewing for a specified period of time. Collagen release rate may be determined by measuring the nitrogen content in the chewing gum before and after chewing, using the procedure described in the examples. In one aspect, the chewing gum of the present disclosure has a collagen release rate of at least 92%, at least 95%, or at least 98% after 12 minutes of chewing. In one particular embodiment, the chewing gum has a collagen release rate of from about 92% to about 99% after 12 minutes of chewing. In one embodiment, at least 375 mg of collagen is released from the chewing gum after chewing 3 pieces of the chewing gum for 12 minutes. In another embodiment, about 411 mg of collagen is released from the chewing gum after chewing 3 pieces of the chewing gum for 12 minutes. Unless otherwise indicated, the collagen release rates disclosed herein are applicable to both the coated chewing gum and the chewing gum center.

### Antioxidants

The chewing gum of the present disclosure further comprises antioxidants. The chewing gum may employ a variety of release mechanisms of the antioxidants, which include delayed, immediate, timed, controlled and variable, alone or in combination using known procedures in the art. In an aspect of the disclosure, the chewing gum may utilize the process of encapsulation as a release mechanism for the antioxidants. Some examples of encapsulation procedures include but are not limited to spray drying, fluid-bed coating, spray chilling, coacervation, agglomeration, fixation, absorption, and entrapment alone or in any combination yielding full or partial encapsulation. The antioxidants may be coated in a two-step process or multiple step process. The materials may be coated with any of the ingredients as described herein to obtain a coated product yielding improved crunch, sensory properties and/or stability.

In one aspect, the chewing gum of the present disclosure includes a water soluble vitamin, such as Vitamin C. Advantageously, Vitamin C helps promote collagen production and helps to maintain healthy skin.

In one aspect, the Vitamin C may be present in the coated chewing gum at a concentration of at least 0.001% by weight, and more particularly, at least 0.01% by weight, or at least 0.1% by weight, or at least 0.2% by weight of the coated chewing gum. In certain embodiments, the Vitamin C is present in the coated chewing gum at a concentration of from about 0.001% to about 20% by weight, from about 0.01% to about 10% by weight, from about 0.1% to about 10% by weight, from about 0.2% to about 5% by weight, or about 0.296% by weight of the coated chewing gum composition. Alternatively, the coated chewing gum may contain Vitamin C in an amount of at least 15 mg/100 g of gum, at least 100 mg/100 g of gum, at least 140 mg/100 g of gum, at least 200 mg/100 g of gum, at least 250 mg/100 g of gum, or at least 290 mg/100 g of gum, including from about 100 mg to 300 mg/100 g of gum, or about 296 mg/100 g of gum.

In one embodiment, the chewing gum is a coated chewing gum comprising a chewing gum center and a coating, wherein the Vitamin C is present in the chewing gum center. In one aspect, the chewing gum center comprises Vitamin C in an amount of at least 0.1% by weight, including at least 0.2% by weight, at least 0.3% by weight, or 0.4% by weight of the chewing gum center. In another aspect, the chewing gum center may contain Vitamin C in an amount of at least 15 mg/100 g of gum center, at least 100 mg/100 g of gum center, at least 140 mg/100 g of gum center, at least 200 mg/100 g of gum center, at least 250 mg/100 g of gum center, or at least 290 mg/100 g of gum center, including from about 100 mg to 300 mg/100 g of gum center, or about 296 mg/100 g of gum center.

The chewing gum of the present disclosure preferably contains Vitamin C in an amount sufficient to provide about 100 mg to about 1000 mg, 400 mg to about 800 mg, or at least 600 mg of vitamin C or more per day to a consumer. In one embodiment, about 115 mg of Vitamin C per 100 g of gum or gum center is released from the gum or gum center after chewing.

Vitamin C as described herein refers to any derivative, compound, or combination of compounds having vitamin C activity. Except where the context demands otherwise, the term “vitamin C” is used generically herein to encompass ascorbic acid, any of its salts, any of its derivatives from natural or artificial sources, including any enantiomer or racemate thereof, and any mixture of such compounds having vitamin C activity.

Non-limiting examples of vitamin C derivatives include calcium ascorbate, magnesium ascorbate, zinc ascorbate, potassium ascorbate, sodium ascorbate, dehydroascorbic acid, L-ascorbic acid 2-0-sulfate, L-ascorbic acid 2-0-phosphate, L-ascorbic acid 3-0-phosphate, L-ascorbic acid 6-hexadecanoate, L-ascorbic acid monostearate, L-ascorbic acid dipalmitate, L-threonic acid, L-xylonic acid, L-lyxonic acid and combinations thereof.

In another aspect, the chewing gum of the present disclosure includes a water insoluble vitamin, such as Vitamin E. Advantageously, Vitamin E has an antioxidant effect, delays cell aging, and helps maintain a youthful appearance (see China National Standard for Nutrition Labeling of Prepacked Food GB 28050-2011, which is herein incorporated by reference).

Vitamin E as described herein encompasses vitamin E acetate, natural and artificial tocopherols, alpha-tocopherol, beta-tocopherol, gamma-tocopherol, delta-tocopherol, combinations or derivatives thereof having vitamin E activity.

In one aspect, the chewing gum of the present disclosure is a coated chewing gum comprising a chewing gum center and a coating, wherein the Vitamin E is in the coating. The Vitamin E may be present in the coated chewing gum of the present disclosure at a concentration at least 0.001% by weight, or at least 0.01% by weight, or at least 0.1% by weight of the coated chewing gum. In one aspect, the Vitamin E is present in the coated chewing gum at a concentration of from about 0.001% to about 10% by weight, from about 0.01% to about 5% by weight, from about 0.1% to about 4% by weight, or about 0.167% by weight of the coated chewing gum. Alternatively, the coated chewing gum contains Vitamin E in an amount of at least 2.1 mg/100 g of gum, at least 70 mg/100 g of gum, at least 100 mg/100 g of gum, at least 120 mg/100 g of gum, or at least 130 mg/100 g of gum. In one embodiment, the coated chewing gum contains Vitamin E in an amount of about 130 mg/100 g of gum.

The coated chewing gum may contain Vitamin E in an amount sufficient to provide about 10.0 mg to 100 mg, 100 mg to about 200 mg, or at least 200 mg of vitamin E or more per day to a consumer. In one embodiment, about 70 mg of Vitamin E per 100 g of gum is released from the gum after chewing.

Optionally, the chewing gum may further comprise alone or in combination, additional vitamins, such as Thiamine (vitamin B1), Riboflavin (vitamin B2), Pyridoxine (Pyridoxal, vitamin B6), Nicotinamide (Niacin), Pantothenic Acid, Biotin, Folic Acid, and/or Cyanocobalamin (vitamin B12).

### Chewing Gum Compositions

The chewing gum compositions of the present disclosure may include the collagen, Vitamin C, and Vitamin E in any of the amounts set forth herein. Chewing gum compositions of the present disclosure may be made using a variety of different compositions that are typically used in chewing gum compositions. Suitable physical forms include sticks, tabs, dragees, chicklets, batons, and the like. Although exact ingredients for each product form will vary from product to product, the specific techniques will be known by one skilled in the art. In general, a chewing gum composition typically contains a chewable gum base portion which is essentially water-insoluble, and a water-soluble bulk portion which includes water soluble bulking agents and other water soluble components as well as flavors and perhaps other active ingredients which are typically water-insoluble. The water-soluble portion dissipates with a portion of the flavor (and other water insoluble actives, if present) over a period of time during chewing. The gum base portion is retained in the mouth throughout the chew.

In one embodiment of the present disclosure, the chewing gum is a coated chewing gum comprising a chewing gum center and a coating. Unless otherwise indicated, the amounts of chewing gum components set forth herein are by weight of the chewing gum center.

The chewing gum of the present disclosure is preferably a coated, sugarless chewing gum containing the collagen, Vitamin C, and Vitamin E. In one embodiment, the chewing gum comprises a coating comprising the Vitamin E, and a chewing gum center comprising the collagen and Vitamin C. The coating may partially or completely cover the surface of the chewing gum center.

The chewing gum center may comprise between approximately 5% to about 95% by weight gum base. Typically, the insoluble gum base may comprise between approximately 10% and about 50% by weight of the chewing gum center, or from approximately 20% to about 40% by weight of the chewing gum center. The present disclosure contemplates employing any commercially acceptable gum base.

The insoluble gum base generally comprises elastomers, elastomer solvents, plasticizers, waxes, emulsifiers, and inorganic fillers. Plastic polymers, such as polyvinyl acetate, which behave somewhat as plasticizers, are also included. Other plastic polymers that may be used include polyvinyl laurate, polyvinyl alcohol, and polyvinyl pyrrolidone. Gum base typically comprises 20 to 40% by weight of the overall chewing gum composition. (i.e., by weight of the chewing gum center). However, in less common formulations it may comprise as low as 5% or as high as 95% by weight.

Synthetic elastomers may include, but are not limited to, polyisobutylene (e.g. having a weight average molecular weight of about 10,000 to about 95,000), butyl rubber (isobutylene-isoprene copolymer), styrene copolymers (having for example a styrene-butadiene ratio of about 1:3 to about 3:1), polyisoprene, polyethylene, vinyl acetate-vinyl laurate copolymer (having for example a vinyl laurate content of about 5% to about 50% by weight of the copolymer), and combinations thereof.

Natural elastomers may include for example natural rubbers such as smoke or liquid latex and guayule, as well as natural gums such as chicle, jelutong, lechi caspi, perillo, sorva, massaranduba balata, massaranduba chocolate, nispero, rosindinha, gutta hang kang and mixtures thereof. Preferred elastomers will depend on, for example, whether the chewing gum in which the base is used is adhesive or conventional, synthetic or natural, bubble gum or regular gum. Elastomers provide the rubbery texture which is characteristic of chewing gum. Elastomers typically make up 5 to 25% by weight of the gum base.

Elastomer solvents which are sometimes referred to as elastomer plasticizers, include but are not limited to natural rosin esters such as glycerol esters, or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerthyritol esters of rosin, synthetics such as terpene resins, polylimonene and other polyterpenes and/or any suitable combination of the forgoing. Elastomer solvents are typically employed at levels of 5 to 30% by weight of the gum base.

Gum base plasticizers are sometimes referred to as softeners (but are not to be confused with water soluble softeners used in the water soluble portion of the gum). Typically, these include fats and oils as well as waxes. Fats and oils are typically vegetable oils which are usually partially or fully hydrogenated to increase their melting point. Vegetable oils suitable for such use include oils of cottonseed, soybean, palm (including palm kernal), coconut, shea, castor, peanut, corn, rapeseed, canola, sunflower, cocoa and others. Less commonly used are animal fats such as milk fat, tallow and lard. Structured fats, which are essentially synthetically compounded glycerol esters (triglycerides) of fatty acids of varying chain lengths, offer an ability to carefully adjust the softening profile by use of short and medium chain fatty acids which are less commonly found in nature. Commonly employed waxes include paraffin, microcrystalline and natural waxes such as beeswax and carnauba. Microcrystalline waxes, especially those with a high degree of crystallinity, may be considered bodying agents or textural modifiers. Plasticizers are typically employed at a level of 5 to 40% by weight of the gum base.

Plastic polymers, such as polyvinyl acetate, which behave somewhat as plasticizers, are also commonly used. Other plastic polymers that may be used include polyvinyl laurate, polyvinyl alcohol, and polyvinyl pyrrolidone. Most gum bases incorporate polyvinyl acetate at a level of 5 to 40% by weight of the gum base.

The gum base typically also includes a filler component. The filler component is typically an inorganic powder such as calcium carbonate, ground limestone, magnesium carbonate, talc, silicate types such as aluminum and magnesium silicate, dicalcium phosphate, tricalcium phosphate, cellulose polymers, such as wood, combinations thereof and the like. The filler may constitute from 5% to about 50% by weight of the gum base. Occasionally, a portion of the filler may be added to the chewing gum mixture separately from the gum base.

Emulsifiers, which may also have plasticizing properties, assist in homogenizing and compatibilizing the different base components. Commonly used emulsifiers include mono- and diglycerides such as glycerol monostearate, lecithin, glycerol triacetate, glycerol monostearate, acetylated monoglycerides, fatty acids and combinations thereof. Emulsifiers are commonly used at a level of 1 to 10% by weight of the gum base.

Gum bases commonly contain optional additives such as other antioxidants and colors which serve their normal functions. Less commonly, flavors and sweeteners may be added to the gum base. These additives, if used, are typically employed at levels of about 1% or less by weight of the gum base.

The water-soluble portion of the chewing gum center may comprise softeners, sweeteners, flavoring agents, and combinations thereof as well as other optional ingredients. For example, the majority of the water soluble portion of the chewing gum center will typically comprise a water-soluble, powdered carbohydrate which serves as a bulking agent. In sugar gums, this most often is sucrose although other sugars such as fructose, erythrose, dextrose (glucose), levulose, tagatose, galactose, trehalose, corn syrup solids and the like, alone or in any combination may also be used.

Generally, sugarless chewing gums will employ sugar alcohols (also called alditols, polyols or polyhydric alcohols) as bulking agents due to their benefits of low cariogenicity, reduced caloric content and reduced glycemic values. Such sugar alcohols include sorbitol, mannitol, xylitol, hydrogenated isomaltulose, maltitol, erythritol, hydrogenated starch hydrolysate solids, and the like, alone or in any combination. Longer chain saccharides such as polydextrose and fructo-oligosaccharides are sometimes employed for their reduced caloric properties and other health benefits. The bulking agents typically comprise approximately 5% to about 95% by weight of the chewing gum center.

Softeners are added to the chewing gum in order to optimize the chewability and mouth feel of the gum. Softeners, also known in the art as plasticizers or plasticizing agents, generally constitute between approximately 0.5% to about 15% by weight of the chewing gum center. These include glycerin, propylene glycol and aqueous sweetener solutions (syrups). Examples of syrups include corn syrups and (generically) glucose syrups which are usually prepared from hydrolyzed starch. For sugarless products, the starch hydrolysate may be hydrogenated to produce an ingredient known as hydrogenated starch hydrolysate syrups or maltitol syrups. These HSH syrups have largely replaced sorbitol solutions previously used in sugarless gums because they also function as binders to improve the flexibility and other physical properties of the gum. Softeners are also often used to control the humectancy (water absorbing properties) of the product.

An emulsifier is sometimes added to the gum to improve the consistency and stability of the gum product. They may also contribute to product softness. Lecithin is the most commonly employed emulsifier, although nonionic emulsifiers such as polyoxyethylene sorbitan fatty acid esters and partial esters of common fatty acids (lauric, palmitic, stearic and oleic acid hexitol anhydrides (hexitans and hexides) derived from sorbitol may also be used. When used, emulsifiers typically comprised 0.5 to 2% by weight of the chewing gum center.

Suitable surface active agents include surface active agents, which can be salts of potassium, ammonium, or sodium. Sodium salts include anionic surface active agents, such as alkyl sulfates, including sodium lauryl sulfate, sodium laureth sulfate, and the like. Other sodium salts include sodium lauroyl sarcosinate, sodium brasslate, and the like. Suitable ammonium salts include betaine derivatives such as cocamidopropyl betaine, and the like.

In the case of sugarless gums, it is usually desirable to add high intensity sweeteners to compensate for the reduced sweetness resulting from substitution of sugar alcohols for the sucrose in sugar gums. More recently, the trend has been to also add high intensity sweeteners to sugar gums to boost and extend flavor and sweetness. High intensity sweeteners (which are sometimes called high potency or artificial sweeteners) may be defined as food acceptable chemicals which are at least twenty times sweeter than sucrose. Commonly used high intensity sweeteners include aspartame, sucralose, and acesulfame-K. Less common are saccharin, thaumatin, alitame, neotame, cyclamate, perilla derived sweeteners, stevia derived sweeteners, monatin, monellin and chalcones.

Usage levels for high intensity sweeteners may vary widely depending on the potency of the sweetener, local market preferences and the nature and level of other ingredients which might impart bitterness to the gum. Typical levels can range from about 0.01% to about 2% by weight, although some applications may dictate usage outside that range. These sweeteners may be combined together, or with non-high intensity sweeteners at varying levels to impart a sweetness synergy to the overall composition.

Flavors can also optionally be employed to impart a characteristic aroma and taste sensation to chewing gum products. As discussed herein, most flavors are water insoluble liquids but water soluble liquids and solids are also known. These flavors may be natural or artificial (synthetic) in origin. Often natural and artificial flavors are combined. It is also common to blend different flavors together in pleasing combinations. Although the range of flavors usable in chewing gums is nearly limitless, they commonly fall into several broad categories. Fruit flavors include lemon, orange, lime, grapefruit, tangerine, strawberry, apple, cherry, raspberry, blackberry, blueberry, banana, pineapple, cantaloupe, muskmelon, watermelon, grape, currant, mango, kiwi and many others as well as combinations. Mint flavors include spearmint, peppermint, wintergreen, basil, corn mint, menthol and others and mixtures thereof. Spice flavors include cinnamon, vanilla, clove, chocolate, nutmeg, coffee, licorice, eucalyptus, ginger, cardamom and many others. Also used are herbal and savory flavors such as popcorn, chili, corn chip and the like. Flavors are typically employed at levels of 0.1 to 4% by weight of the finished gum product (e.g., coated chewing gum). In recent years there has been a trend toward increasing flavor levels to provide higher flavor impact.

It is common to co-dry and encapsulate flavors with various carriers and/or diluents. For example, spray-dried flavors using gum Arabic, starch, cyclodextrin or other carriers are often used in chewing gum for protection, controlled release, control of product texture and easier handling as well as other reasons. When flavors are in such forms, it will often be necessary to increase the usage level to compensate for the presence of the carriers or diluents.

The chewing gum of the present disclosure may employ various sensates. Generally, sensates may be any compounds that cause a cooling, heating, warming, tingling or numbing, for example, to the mouth or skin. Cooling agents are trigeminal stimulants that impart a cool sensation to the mouth, throat and nasal passages. The most widely known cooling agent is menthol, although this is often considered a flavor due to its aroma properties and the fact that it is a natural component of peppermint oil. More often, the term cooling agent refers to other natural or synthetic chemicals used to impart a cooling sensation with minimal aroma. Commonly employed cooling agents include ethyl p-menthane carboxamide and other N-substituted p-menthane carboxamides, N,2,3-trimethyl-2-isopropyl-butanamide and other acyclic carboxamides, menthyl glutarate (Flavor Extract Manufacturing Association (FEMA 4006)), 3-1-menthoxypropane-1,2-diol, isopulegol, menthyl succinate, menthol propylene glycol carbonate, menthol ethylene glycol carbonate, menthyl lactate, menthyl glutarate, menthone glyceryl ketal, p-menthane-1,8-diol, menthol glyceryl ether, N-tertbutyl-p-menthane-3-carboxamide, p-menthane-3-carboxylic acid glycerol ester, methyl-2-isopryl-bicyclo (2.2.1), heptane-2-carboxamide, menthol methyl ether and others and combinations thereof.

Cooling agents may be employed to enhance the cool taste of mint flavors or to add coolness to fruit and spice flavors. Cooling agents also provide the perception of breath freshening, which is the basis of the marketing of many chewing gums and confections.

Trigeminal stimulants other than cooling agents may be employed in the chewing gums of the present disclosure. These include warming agents such as capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin, menthol glycerin ether and unsaturated amides and tingling agents such as Jambu extract, vanillyl alkyl ethers such as vanillyl n-butyl ether, spilanthol, Echinacea extract and Northern Prickly Ash extract. Some of these components are also used as flavoring agents.

Chewing gum generally conveys oral care benefits. In addition to mechanical cleaning of the teeth provided by the chewing action, saliva stimulated by chewing, flavor and taste from the product conveys additional beneficial properties in reducing bad breath, neutralizing acid, and the like. Saliva also contains beneficial polypeptides and other components which may improve the oral environment. These include: antimicrobial proteins, such as lysozyme, lactoferrin, peroxidases, and histatins; inhibitors of spontaneous crystallization, such as statherin.

The chewing gums of the present disclosure can provide these benefits along with the benefits disclosed herein, and may also be used as vehicles for the delivery of specialized oral care agents. These may include antimicrobial compounds such as Cetylpyridinium Chloride (CPC), triclosan and chlorhexidine; anti-caries agents such as calcium and phosphate ions, plaque removal agents such as abrasives, surfactants and compound/ingredients; plaque neutralization agents such as ammonium salts, urea and other amines; anti-tartar/calculus agents such as soluble pyrophosphates salts; anti halitosis agents such as parsley oil and copper or zinc salts of gluconic acid, lactic acid, acetic acid or citric acid, and whitening agents such as peroxides; agents that may provide either local or systemic anti-inflammatory effects to limit gingivitis, such as COX-2 inhibitors; agents that may reduce dentinal hypersensitivity, such as potassium salts to inhibit nerve cell transmission, and calcium phosphate salts to block the dentinal tubules.

Certain flavors such as peppermint, methyl salicylate, thymol, eucalyptol, cinnamic aldehyde and clove oil (eugenol) may have antimicrobial properties which benefit the oral cavity. These flavors may be present primarily for flavoring purposes or may be added specifically for their antimicrobial properties.

Certain mineral agents may contribute to dental health, in addition to ones disclosed by the disclosure by combating demineralization and enhancing remineralization of teeth. Such ingredients include fluoride salts, dental abrasives and combinations thereof

Teeth color modifying substances may be considered among the oral care actives useful. These substances are suitable for modifying the color of the teeth to satisfy the consumer such as those listed in the CTFA Cosmetic Ingredient Handbook, 3rd Edition, Cosmetic and Fragrances Associations Inc., Wash. D.C. (1982), incorporated herein by reference. Specific examples include talc, mica, magnesium carbonate, magnesium silicate, aluminum magnesium carbonate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder and mixtures thereof.

The chewing gums of the present disclosure may be used to deliver biologically active agents and other beauty ingredients to the chewer. Such ingredients include vitamins, minerals, anti-oxidants, nutritional supplements, dietary supplements, functional food ingredients (e.g., probiotics, prebiotics, lycopene, phytosterols, stanol/sterol esters, omega-3 fatty acids, adenosine, lutein, zeaxanthin, grape seed extract, Ginkgo biloba, isothiocyanates and the like), OTC and prescription pharmaceuticals, vaccines, and nutritional supplements.

It may be desirable to take certain steps to increase or decrease the rate of the release of the agent or to ensure that at least a minimum quantity is released. Such measures as encapsulation, isolation of the active, measures to increase or decrease interaction with the water-insoluble portion of the gum and enteric coating of actives may be employed to that end.

In general, chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent. Further parts of the bulking agent are added to the mixer. The collagen may be added with the bulking agent and/or with any high intensity sweeteners. The Vitamin C and any additional flavoring agents are typically added with the final portion of the bulking agent or with the high intensity sweeteners. Other optional ingredients are added to the batch in a typical fashion, well known to those of ordinary skill in the art.

The entire mixing procedure typically takes from five to fifteen minutes, but longer mixing times may sometimes be required. Those skilled in the art will recognize that many variations of the above described procedure may be followed.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacturing involves the dispersive (often high shear) mixing of difficult-to-blend ingredients, such as elastomer, filler, elastomer plasticizer, base softeners/emulsifiers, and sometimes waxes. This process typically requires long mixing times. Chewing gum product manufacture also involves combining the gum base with more delicate ingredients such as product softeners, bulk sweeteners, high intensity sweeteners and flavoring agents using distributive (generally lower shear) mixing, for shorter periods.

Chewing gums of the present invention are also coated. Pellet or ball gum is prepared as conventional chewing gum, but formed into pellets that are pillow shaped, or into balls. The pellets/balls can be then sugar coated or panned by conventional panning techniques to make a unique sugar coated pellet gum. As discussed herein, the Vitamin E is incorporated into the chewing gum coating.

Conventional panning procedures generally coat with sucrose, but recent advances in panning have allowed the use of other carbohydrate materials to be used in the place of sucrose. Some of these components include, but are not limited to, dextrose, maltose, palatinose, xylitol, lactitol, hydrogenated isomaltulose and other new alditols or a combination thereof. These materials may be blended with panning modifiers including, but not limited to, gum arabic, maltodextrins, corn syrup, gelatin, cellulose type materials like carboxymethyl cellulose or hydroxymethyl cellulose, starch and modified starches, vegetable gums like alginates, locust bean gum, guar gum, and gum tragacanth, insoluble carbonates like calcium carbonate or magnesium carbonate and talc. Antitack agents may also be added as panning modifiers which allow the use of a variety of carbohydrates and sugar alcohols to be used in the development of new panned or coated gum products. Essential oils may also be added with the sugar coating to yield unique product characteristics.

The following examples are illustrative of preferred embodiments of the disclosure and are not to be construed as limiting. All percentages are based on the percent by weight of the composition unless otherwise indicated, and all totals equal 100% by weight.

### Example 1: Chewing Gum Formulations

Chewing gum formulations were prepared with and without collagen as shown below in Tables 1-3.

TABLE 1
Chewing Gum Center Formula¹

| Ingredients | Percent range |
|---|---|
| Gum base | 30-33 |
| Bulk sweeteners | 33.5-64.8 |
| Softeners | 1-4 |
| Acids | 0.2-0.6 |
| High Intensity Sweeteners | 1-1.9 |
| Flavor | 1-2 |
| Collagen | 0-25 |
| Total | 100 |

¹Serving size for prototypes is 6 pieces with a piece size of 2.1 g. Collagen content from 1000 to 3200 mg.

TABLE 2
Factory Formula

| Layer | Ingredients | Percent Range |
|---|---|---|
| Gum Layer | Gum Base | 32.3-41.25 |
| | Bulk Sweetener | 31.25-58 |
| | High Intensity Sweeteners | 1-2.5 |
| | Softener | 0-4 |
| | Acids | 1-2.5 |
| | Flavor | 1-2.5 |
| | Vitamin C | 0.2-1 |
| | Collagen | 7-15 |
| | Total | 100 |
| Coating layer | Coating Syrup | 96-98.8 |
| | Flavor + Vitamin E | 1.5-2.5 |
| | Coating Wax | 0.2-0.5 |
| | Total | 100 |

### Example 2: Shelf-Life Study

The stability of the chewing gum compositions of Table 1 in various packaging was assessed over a period of 8 weeks at 30° C./70% relative humidity (RH) and compared to a control using the following scale:
Green: Identical or similar to control/reference
Yellow: Acceptable but moderately different to control/reference
Red: Greatly different from control and has a gross defect

The results are shown below in Table 3.

TABLE 3

| Gum Type | Container | Assessment on | Time Zero | 4 weeks | 8 weeks |
|---|---|---|---|---|---|
| Control | Plastic bottle with high barrier | Appearance, texture, and flavor | Green | Green | Green |
| Collagen - Low amount | Plastic bottle with high barrier | Appearance, texture, and flavor | Green | Green | Green |
| Collagen - High amount | Plastic bottle with high barrier | Appearance, texture, and flavor | Green | Green/Yellow | Green/Yellow |
| Control | Plastic bottle with low barrier | Appearance, texture, and flavor | Green | Green | Green |
| Collagen - Low amount | Plastic bottle with low barrier | Appearance, texture, and flavor | Green | Green | Green |
| Collagen - High amount | Plastic bottle with low barrier | Appearance, texture, and flavor | Green | Green/Yellow | Green/Yellow |

### Example 3: Collagen Chew-Out Rate

The chew out rate (also referred to herein as release rate) of a collagen-containing coated chewing gum composition of the disclosure was assessed by determining the nitrogen content remaining after a 12 minute and 20 minute chew-out of a fresh sample. Every 100 g of the sample being chewed contributed 40.79 g of gum cud weight. The coated chewing gum comprised collagen in an amount of 17% by weight of the chewing gum center. The results are summarized below in Tables 4 and 5.

TABLE 4

| | Sample 1 | Sample 2 | Sample 3 |
|---|---|---|---|
| | (fresh sample) | (12 m chew-out) | (20 m chew-out) |
| Nitrogen content | 1.25 | 0.404 | 0.243 |
| (g/100 g) | | | |

TABLE 5

| | Nitrogen content | Nitrogen content |
|---|---|---|
| | (collagen) | (collagen) |
| | Sample 2 | Sample 3 |
| Chew-out rate | (1.25-0.404*40.79/ | (1.25-0.243*38.91/ |
| (12 min) | 100)/1.25 | 100)/1.25 |
| | 98.4% | 92.43% |
| Intake content | 1.25*98.4%*5.55 = | 1.25*92.43%*5.55 = |
| (12 min) | 6.83 g/100 g | 6.41 g/100 g |
| | (collagen) | (collagen) |
| Intake content/ | 461 mg (collagen) | 431 mg (collagen) |
| 3 pieces (12 min) | | |
| Fortification requirement | 375 mg | 375 mg/3 pieces |

The average collagen chew-out rate was determined to be 95.42%.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A coated chewing gum comprising:
Vitamin C;
Vitamin E; and
collagen in an amount of from about 6% to about 20% by weight of the coated chewing gum.

2. The coated chewing gum of claim 1, wherein the coated chewing gum comprises collagen in an amount of from about 6.1% to about 18.5% by weight of the coated chewing gum.

3. The coated chewing gum of claim 1, wherein the coated chewing gum comprises Vitamin C in an amount of at least 0.2% by weight of the coated chewing gum.

4. The coated chewing gum of claim 1, wherein the coated chewing gum comprises Vitamin C in an amount of about 0.296% by weight of the coated chewing gum.

5. The coated chewing gum of claim 1, wherein the coated chewing gum comprises Vitamin E in an amount of at least 0.1% by weight of the coated chewing gum.

6. The chewing gum of claim 1, wherein the coated chewing gum comprises Vitamin E in an amount of about 0.167% by weight of the chewing gum.

7. The coated chewing gum of claim 1, wherein the coated chewing gum comprises a coating and a chewing gum center.

8. The coated chewing gum of claim 7, wherein the chewing gum center comprises collagen in an amount of from about 8% to about 25% by weight of the chewing gum center.

9. The coated chewing gum of claim 8, wherein the chewing gum center comprises collagen in an amount of from about 8.33% to about 25% by weight of the chewing gum center.

10. The coated chewing gum of claim 7, wherein the chewing gum center comprises Vitamin C in an amount of at least 0.1% by weight of the chewing gum center.

11. The coated chewing gum of claim 7, wherein the chewing gum center comprises Vitamin C in an amount of about 0.4% by weight of the chewing gum center.

12. The coated chewing gum of claim 7, wherein the coating comprises Vitamin E.

13. The coated chewing gum of claim 1, wherein the coated chewing gum is a sugarless chewing gum.
